(19) United States
(12) Patent Application Publication
(10) Pub. No.: US 2020/0147224 A1
(43) Pub. Date: May 14, 2020

(54) COMPOSITION CONTAINING CATIONIC HYDROXYETHYL CELLULOSE

(71) Applicants: ROHM AND HAAS COMPANY, Collegeville, PA (US); DOW GLOBAL TECHNOLOGIES LLC, Midland, MI (US)

(72) Inventors: Joshua Katz, Midland, MI (US); Yujing Tan, Midland, MI (US); Emmett M Partain, III, Midland, MI (US); Demetrius Dielman, Midland, MI (US); Susan Jordan, Midland, MI (US); Jaime Curtis-Fisk, Midland, MI (US); Ryan DePuit, Midland, MI (US)

(21) Appl. No.: 16/743,162

(22) Filed: Jan. 15, 2020

Related U.S. Application Data
(62) Division of application No. 15/563,020, filed on Sep. 29, 2017, now abandoned.
(60) Provisional application No. 62/142,045, filed on Apr. 2, 2015.

(51) Int. Cl. A61K 47/38; A61K 9/08; C08L 1/28; C08B 11/193; C08B 11/14; A61K 9/00

(52) U.S. Cl. CPC A61K 47/38; A61K 9/08; A61K 9/0043; C08B 11/193; C08B 11/14; C08L 1/288

(57) ABSTRACT

An aqueous solution comprising a cationic polymer dissolved in water, wherein said cationic polymer comprises a hydrophobic quaternary ammonium group covalently attached to a hydroxyethyl cellulose polymer backbone. Also, a method of delivering a drug to a mucosal surface in a living body, said method comprising applying the aqueous solution to said mucosal surface.

### CROSS-REFERENCE TO RELATED APPLICATION

This application is a division of application Ser. No. 15/563,020 which represents a national filing under 35 U.S.C. 371 of International Application No. PCT/US16/024491 filed Mar. 28, 2016, and claims priority of U.S. Provisional Application No. 62/142,045 filed Apr. 2, 2015, the contents of all prior applications are incorporated herein by reference in their entirety for all purposes.

Mucosal surfaces line various cavities in a living body, including those exposed to the external atmosphere. Mucosal surfaces are involved in absorption of compounds into the body. Consequently a useful method of introducing a physiologically active agent into the body is to apply a composition containing the physiologically active agent to the mucosal surface. When applying such a composition to a mucosal surface, it is desirable that the composition reside on the mucosal surface for a relatively long time. It is also desirable that the composition allow the physiologically active agent to readily permeate through the mucosal surface so that the physiologically active agent can enter the tissues and/or the bloodstream of the living body. Often, a composition that contains a physiologically active agent also contains one or more additional compound called “excipients.” It is desirable that an excipient improve the residence time of the composition and/or improves the permeation of the physiologically active agent through the mucosal surface. An important mucosal surface in the human body for introduction of physiologically active agents is the mucosal surface inside the nasal cavity.

U.S. Pat. No. 3,472,840 describes certain specific cationic polysaccharide polymers. It is desired to provide cationic polysaccharide polymers that provide improved permeation through mucosal surfaces compared to those described by U.S. Pat. No. 3,472,840. In the past, chitosan has been used as an excipient. Chitosan is derived from a natural product and therefore is subject to variability in quality and characteristics; also, the usefulness of chitosan is undesirably limited because chitosan is not soluble at certain desirable pH values. It is desired to provide a synthetic polymer that performs well as an excipient and/or that is soluble over a desirably wide range of pH values.

The following is a statement of the invention.

The first aspect of the present invention is an aqueous solution comprising a cationic polymer dissolved in water, wherein said cationic polymer comprises a hydrophobic quaternary ammonium group covalently attached to a hydroxyethyl cellulose polymer backbone.

A second aspect of the present invention is a method of delivering a drug to a mucosal surface in a living body, said method comprising applying the aqueous solution of the first aspect to said mucosal surface, wherein said aqueous solution of the first aspect comprises said drug.

The following is a detailed description of the invention.

As used herein, the following terms have the designated definitions, unless the context clearly indicates otherwise.

Mucosal surfaces are found in living bodies of animals and humans. Mucosal surfaces contain epithelium, which produces and excretes mucus. Examples of mucosal surfaces are found in the nasal cavity, the mouth, the eye, the ear, the vagina, the esophagus, the stomach, the intestines, and other parts of the body.

A compound is considered herein to be cationic if an atom or a chemical group that bears a positive charge is covalently bound to the compound. A cationic functional group is an atom or a chemical group that bears a positive charge. The cationic functional group bears a positive charge at all pH values over a range that includes the range of 4 to 11.

An amount of polymer is considered herein to be dissolved in water if the mixture of that amount of the polymer and water forms a composition that at 25° C. is homogeneous and that does not show phase separation of the polymer from the water at 25° C.

As used herein, a drug is a compound having beneficial prophylactic and/or therapeutic properties when administered to an individual, typically a mammal, especially a human individual.

As used herein, a hydrophobic group is a chemical group that contains a group of 8 or more carbon atoms, where the carbon atoms are connected to each other in a manner that is linear, cyclic, branched, or a combination thereof, and where the only atoms in the hydrophobic group are carbon and hydrogen.

As used herein, a hydrophobic quaternary ammonium group is a chemical group having the structure I

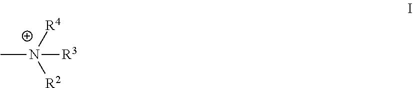

where —R² and —R³ are substituted or unsubstituted hydrocarbon groups each containing one or more carbon atom, and —R⁴ contains one or more hydrophobic group. A non-hydrophobic quaternary ammonium group is a chemical group having structure I in which none of —R², —R³, and —R⁴ contains a hydrophobic group.

The present invention involves a cationic polymer that contains a cationic functional group attached to a hydroxyethyl cellulose polymer backbone. That is, the cationic polymer has a structure that would result if a molecule of the hydroxyethyl cellulose polymer (the “backbone” polymer) were subjected to one or more chemical reactions to replace one or more of the hydroxyl groups on the hydroxyethyl cellulose polymer with cationic functional groups. Regardless of the method of making the cationic polymer, the cationic polymer can be characterized by the properties of the backbone polymer.

Hydroxyethyl cellulose (HEC) polymer has repeat units of the structure II:

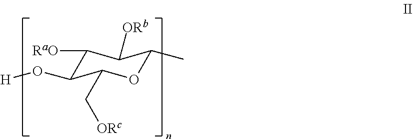

In structure II, the repeat unit is shown within the brackets. The degree of polymerization (n) is 10 or higher and is sufficiently large that structure II is a polymer; that is, when n is large enough, the 2% standard solution viscosity (as defined below) of the HEC will be 10 mPa·s or higher. —R^{a}, —R^{b}, and —R^{c} is each —[CH₂CH₂O]ₓ—H, where each x is chosen from 0, 1, 2, 3, or 4. The choice of —R^{a}, —R^{b}, and —R^{c} may be the same in each repeat unit, or different repeat units may have different choices of —R^{a}, —R^{b}, and —R^{c}. One or more repeat units has one or more of —R^{a}, —R^{b}, and —R^{c} in which x is from 1 to 4.

The cationic polymer of the present invention has repeat units of the structure II in which one or more of —R^{a}, —R^{b}, and —R^{c} has the structure —[CH₂CH₂O]ₓ—R¹, where R¹ has structure III:

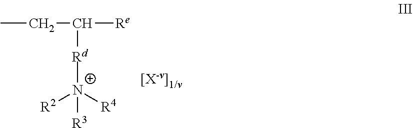

where —R^{d}— is a bivalent organic group, and —R^{e} is either a hydrogen atom or a hydroxyl group. Preferably, —R^{d}— is a hydrocarbon group with 0 to 8 carbon atoms; more preferably with 1 to 2 carbon atoms; more preferably with 1 carbon atom. Preferably, —R^{e} is a hydroxyl group. —R², —R³, and —R⁴ is each independently a substituted or unsubstituted hydrocarbon group. Preferably —R² and —R³ are unsubstituted hydrocarbon groups; more preferably —R² and —R³ are alkyl groups. Preferably —R² and —R³, is each independently an alkyl group with 3 or fewer carbon atoms; more preferably and alkyl group with 2 or fewer carbon atoms; more preferably a methyl group. —R⁴ is a chemical group containing a hydrophobic group. Preferably, —R⁴ is an alkyl group. Preferably, —R⁴ has 10 or more carbon atoms; more preferably 12 or more carbon atoms. Preferably, —R⁴ has 18 or fewer carbon atoms; more preferably 16 or fewer carbon atoms; more preferably 14 or fewer carbon atoms; more preferably 12 or fewer carbon atoms. Preferably, one or more —R^{b} or —R^{c} group has structure II. X^{-v} is an anion of valence v. It is contemplated that if v is greater than 1, then v groups of structure III will be associated with each anion of valence v. Preferred anions are halide ions; more preferred is chloride ion.

Preferably, the cationic polymer of the present invention either has no non-hydrophobic quaternary ammonium groups or else, if any non-hydrophobic quaternary ammonium groups are present, there are very few non-hydrophobic quaternary ammonium groups. Specifically, it is preferred that the mole ratio of non-hydrophobic quaternary ammonium groups to hydrophobic quaternary ammonium groups is 0:1 to 0.1:1; more preferably 0:1 to 0.01:1; more preferably 0:1.

The cationic polymer of the present invention may be characterized by the viscosity of a solution of either 1% or 2% by weight of the cationic polymer in water at 25° C. That viscosity is measured at 25.0° C. and a shear rate of 6.31 sec⁻¹ using a TA Instruments DHR-3 rheometer equipped with either a stainless steel cone and plate sensor (60 mm diameter and 0.5° cone angle) or a concentric cylinder cup and bob sensor. If the viscosity of the 2% solution is greater than 18,000 mPa·s, then a solution of 1% by weight of the cationic polymer in water is made and tested by the same method at 25° C. The result of this viscosity testing is reported herein as the “standard solution viscosity.”

Preferably, the standard solution viscosity of the cationic polymer of the present invention in a 2% by weight solution is 50 mPa·s or higher; more preferably 100 mPa·s or higher. Preferably, the standard solution viscosity of the cationic polymer of the present invention in a 1% by weight solution is 30,000 mPa·s or lower. Preferably, the standard solution viscosity of the cationic polymer of the present invention in a 2% by weight solution is 18,000 mPa·s or lower; more preferably 10,000 mPa·s or lower.

The cationic polymer of the present invention may be characterized by the weight-average molecular weight (Mw), which is measured by gel permeation chromatography. Preferably, Mw is 50,000 or higher; more preferably 100,000 or higher; more preferably 200,000 or higher. Preferably, Mw is 1,000,000 or lower; more preferably 500,000 or lower.

The cationic polymer of the present invention may be characterized by the cationic degree of substitution (CDS), which is defined as the molar ratio of repeat anhydroglucose units with the structure III over total repeat anhydroglucose units. The CDS is measured and calculated from Kjeldahl nitrogen analysis. Preferably, the cationic degree of substitution is 0.01 or higher; more preferably 0.02 or higher; more preferably 0.05 or higher.

A preferred method of making the cationic polymer is to react a hydroxyethyl cellulose polymer with a compound of structure IV, V, or VI:

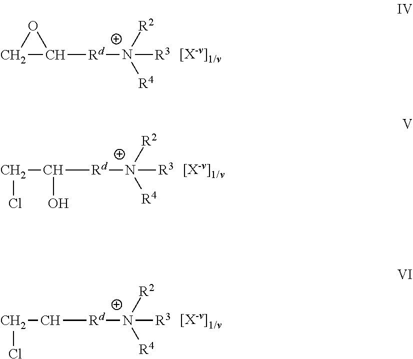

where the R^{d}, R², R³, R⁴, X, and v are defined above.

The present invention involves a solution that contains a cationic polymer dissolved in water. Preferably, the amount of water in the solution, by weight based on the total weight of the volatile components in the solution, is 50% or more; more preferably 75% or more; more preferably 90% or more.

The amount of the cationic polymer in the solution is preferably, by weight based on the weight of the solution, 0.01% or more; more preferably 0.1% or more. The amount of polymer in the solution is preferably, by weight based on the weight of the solution, 10% or less; 5% or less; more preferably 2% or less; more preferably 1% or less.

The solution optionally contains additional ingredients such as, for example, surfactants, thickeners, buffers, pH adjusters, preservatives, and mixtures thereof

Preferably the solution is a buffer solution that contains inorganic salts. One preferred buffer solution is phosphate buffered saline (PBS) solution, which contains sodium chloride and a sodium salt of a phosphorous-containing anion, and optionally also contains potassium chloride and a potassium salt of a phosphorous-containing anion.

The solution may be a liquid, a gel, a lotion, a cream, or another form. Preferred is a liquid. Preferably the viscosity of the solution, as measured by steady shear viscometry using cone and plate at 10 sec⁻¹ at 25° C., is 1,000 mPa·s or less; more preferably 300 mPa·s or less; more preferably 100 mPa·s or less; more preferably 30 mPa·s or less; more preferably 10 mPa·s or less.

Preferred mucosal surfaces are the mucosal surfaces of the nasal cavity, the mouth, the eye, the ear, the vagina, the esophagus, the stomach, the intestines, and combinations thereof more preferred are the mucosal surfaces of the nasal cavity.

Preferably the composition of the present invention contains one or more physiologically active agents, preferably one or more physiologically active agents selected from the following: one or more drugs; one or more diagnostic agents; or one or more essential oils; or one or more physiologically active agents that are useful for cosmetic or nutritional purposes. Preferred physiologically active agents are drugs. Preferred drugs are soluble or dispersible in water at 15° C. to 40° C., in concentrations that are therapeutically useful. Preferred drugs that, in the absence of an effective excipient, have undesirably low capability of absorption into the body through a mucosal surface.

Physiologically active agents that are useful for intranasal delivery are known in the art. Some physiologically active agents and some methods of intranasal delivery are described in WO 2015/009799.

The composition of the present invention is particularly useful for intranasal delivery of one or more physiologically active agents or for delivery through a mucosal membrane located in the nasal cavity, such as drugs utilized in therapies for allergic rhinitis, nasal congestion and infections, in treatments of diabetes, migraine, nausea, smoking cessation, acute pain relief, nocturnal enuresis, osteoporosis, vitamin B-12 deficiency, and for administering intranasal vaccine such as, for example, influenza vaccine; however, the physiologically active agents are not limited to these examples. Especially preferred drugs are acetaminophen, azelastine hydrochloride, beclomethasone dipropionate monohydrate, sumatriptan succinate (SS), dihydroergotamine mesylate, fluticasone propionate, triamcinolone acetonide, budesonide, fentanyl citrate, butorphanol tartrate, zolmitriptan, desmopressin acetate hydrate, salmon calcitonin, nafarelin acetate, buserelin acetate, elcatonin, oxytocin, insulin, mometasone furoate, estradiol, metoclopramide, xylometazoline hydrochloride, ipratropium bromide hydrate, olopatadine hydrochloride, oxymetazoline hydrochloride, dexpanthenol, hydrocortisone, naphazoline hydrochloride, phenylephrine hydrochloride, mepyramine maleate, phenylephrine hydrochloride, cromolyn sodium, levocabastine hydrochloride, vitamin B12, prednisolone sodium metasulphobenzoate, naphazoline nitrate, tetrahydrozoline hydrochloride, chlorpheniramine maleate, benzethonium chloride, ketotifen fumarate, histamine dihydrochloride, fusafungine, or combinations thereof. Examples of essential oils are menthol, methyl salicylate, thymol, eucalyptus oil, camphor, anise, sweet orange, or combinations thereof

The following are examples of the present invention. Example numbers starting with “C” denote comparative examples.

The following comparative polymers were tested:
Comp1=SOFTCAT™ SX-1300H, from the Dow Chemical Company, contains both hydrophobic and non-hydrophobic cationic groups. The mole ratio of non-hydrophobic cationic groups to hydrophobic cationic groups is larger than 5:1.
Comp2=UCARE™ KG-30M, quaternized HEC, from the Dow Chemical Company; has no hydrophobic group.
Comp3=UCARE™ JR-400 polymer, cationic HEC from the Dow Chemical Company; has no hydrophobic group.
Chitosan=naturally-derived amine-functional polymer; does not have hydrophobic groups.

The following abbreviations are used.
TEER=trans-endothelial electrical resistance
API=active pharmaceutical ingredient, a type of physiologically active agent
SS=sumatriptan succinate (a drug)
PBS=phosphate buffer saline solution

The following Example polymers were used. Each Example polymer is described by structure II above, where —R^{a} and —R^{b} are —H; where —R^{c} is —[CH₂CH₂O]ₓ—R¹, where some repeat units have x=1 or 2; where —R¹ has structure III above, where —R^{d} is —CH₂—, where —R² and —R³ are methyl, and where —R⁴ is an alkyl hydrophobic group. The Example polymers were as follows:

Example Polymers

| Polymer | MW | CDS⁽³⁾ | Viscosity⁽⁴⁾ | %⁽⁵⁾ | Phobe⁽⁶⁾ |
|---|---|---|---|---|---|
| P1 | 275,000 | 0.0784 | 8617 | 2 | 12 |
| P2 | 275,000 | 0.0191 | 8833 | 2 | 18 |
| P3 | 1,000,000 | 0.131 | 6719 | 2 | 12 |
| P4 | 1,600,000 | 0.0257 | 6035 | 1 | 18 |
| P5 | 1,600,000 | 0.079 | 17058 | 2 | 12 |
| P6 | 1,000,000 | 0.075 | 11634 | 1 | 12 |
| P7 | 280,000 | 0.078 | 207 | 2 | 12 |
| P8 | 1,600,000 | 0.14 | 6400 | 1 | 12 |

⁽³⁾Cationic degree of substitution
⁽⁴⁾viscosity of solution in water at 25° C., mPa*s
⁽⁵⁾concentration of polymer in viscosity test solution, weight %
⁽⁶⁾number of carbon atoms in −R⁴

EpiAirway™ Tissue Models, 0.2% TRITON™ X-100 surfactant, and 3-(4,5-dimethylthiazol-2-yl)-2,5-diphenyltetrazolium bromide (MTT) assays were acquired from MatTek Corporation. After receiving tissues (24), they were removed from the agar media, moved to clean 6-well plates containing 0.9 mL of fresh Assay Media, and cultured overnight (eighteen hours) in a sterile environment according to the product information. The Assay Media was composed by base medium (Dulbecco's Modified Eagle's Medium), growth factors/hormones (epidermal growth factor, insulin, hydrocortisone and other stimulators of epidermal differentiation), antibiotics (gentamicin 5 μg/mL) and anti-fungal agent (amphotericin B 0.25 μg/mL).

Tissues were removed from the incubator (37° C., 5% CO₂) and prepared for a media exchange. Media exchange was performed on all tissues before being returned to the incubator. After 1-2 hours of additional incubation, twelve tissues were removed from the incubator, media was discarded, and tissues were rinsed with Phosphate Buffer Saline (PBS) (from Dulbecco) and tested for TEER before being used for time dependent studies of permeation of API). TEER measurement was performed using an Endohm chamber coupled with EVOM²™ resistance meter from World Precision Instruments Company. Briefly, each tissue was placed into the Endohm chamber and covered by a cup with electrode, and reading shown on the resistance meter was recorded. Following TEER, tissues were moved to pre-labeled clean 24-well plates containing 250 μL of fresh media in each well (4 or 6 tissues per plate to allow for full permeation study). The remaining twelve tissues were left in the incubator for an additional 24 hours of incubation, to be used for additional permeation studies on the following day.

Donor solutions were prepared 16-20 hours prior to use. The donor solutions were prepared in the following manner: predetermined concentrations/amounts of excipient (surfactant or polymer) were analytically weighed into 50 mL conical tubes, diluted with an appropriate amount of PBS, placed on a rocking shaker for approximately 4 hours at room temperature (approximately 23° C.) and allowed to hydrate. After hydration, the donor solutions were sterile filtered using Steriflip® filter units from Millipore and stored at 4° C. until use.

All donor solutions had 2 mg/mL SS, except for Comparative Example 1, which used 50 mg/mL of SS.

Permeation studies were carried out in the following manner: donor solutions (100 μL) were carefully pipetted onto the apical surface of their respective tissues and incubated at 37° C., 5% CO₂ for 5 minutes (5 minute time point). After incubation, each tissue was moved to a new well with fresh media. Receiver solution from the previous well was collected and placed in a pre-labeled Waters Total Recovery LC/GC vial on dry ice. The tissues were then returned to the incubator for an additional 10 minute incubation period (15 minute time point). After incubation, the process of moving the tissues to the next well, collection of the permeated receiver solutions and incubation was repeated for additional time points up to 240 minutes. Following the 240 minute experimental period, the remaining donor solution on the apical surface of the tissue was collected and placed on dry ice, tissues were rinsed with PBS, and the final TEER measurements were taken.

Following the final TEER measurements, tissue percent viability was measured using the MTT Assay. This kit was used to indirectly measure the amount of nicotinamide adenine dinucleotide phosphate (NADPH) produced by the cells by measuring optical density of the formazan at 570 nm. A positive correlation of NADPH amount and cell viability is known. The cells treated with PBS buffer only were set to 100% which was used for normalization of all other samples. The result reported (“Viability %”) is the quotient obtained by dividing the optical density at 570 nm for a sample by the optical density at 570 nm of the PBS buffer only, for the same optical path length.

Donor and receiver solution samples were removed from storage at −80° C., thawed on ice, and analyzed using the HPLC method developed for Sumatriptan Succinate. Agilent 1100 serial binary gradient liquid chromatograph system was used. Details as following:
Column: Waters SunFire C18, 3.5 μm, 3.0×100 mm, Lot No 150331961
UV Detector Wavelength: 280 nm
Eluent A: 0.1% Trifluoroacetic acid in DI Water
Eluent B: 0.1% Trifluoroacetic acid in 1-Propanol
Gradient: 2% B to 100% B in 20 minutes, hold 100% B for 1 minute, 100% B to 2% B in 1 minute.
Post Run Time: 5 minutes
Flow Rate: 0.4 mL/min
Column Temperature Setting: 27° C.

From the solutions collected at the various times, the effective permeation coefficient (Peff) was calculated by

Peff=(TSS)/[(MA)*(DC)*(PT)]

where
TSS=total amount of SS in permeation (in units of mg)
MA=membrane area (equaled 0.6 cm²)
DC=donor concentration (in units of mg/cm³)
PT=permeation time (equaled 14400 sec)

Comparative Example 1: Permeation of sumatriptan succinate (SS) in PBS buffer with various comparative cationic polymers in the solution. Results were as follows:

Permeation of SS

| Example: | C1-1 | C1-2 | C1-3 | C1-4 | C1-5 | C1-6 | C1-7 |
|---|---|---|---|---|---|---|---|
| Polymer: | none | Comp1 | Comp1 | Comp2 | Comp3 | Comp3 | Chitosan |
| Conc⁽¹⁾ | 0 | 0.5% | 0.1% | 0.1% | 0.5% | 0.1% | 0.1% |
| N⁽²⁾ | 2 | 3 | 2 | 3 | 2 | 2 | 2 |
| Remain⁽³⁾ | 62.1 | 51.5 | 55.7 | 65.8 | 52.5 | 64.2 | 38.9 |
| Perm⁽⁴⁾ | 2.0 | 2.2 | 1.8 | 2.3 | 1.4 | 1.8 | 37.6 |
| Peff⁽⁵⁾ | 0.23 | 0.25 | 0.21 | 0.27 | 0.16 | 0.21 | 4.4 |

⁽¹⁾% weight of polymer on total weight of solution
⁽²⁾number of replicate samples tested. Results shown are averages over the replicates
⁽³⁾weight % of SS remaining in donor solution based on total SS
⁽⁴⁾weight % or SS permeated through tissue based on total SS
⁽⁵⁾units are 10⁻⁶ cm/sec (for example, Peff of C1-1 was 0.23 × 10⁻⁶ cm/sec)

All of the synthetic polymers tested (Comparative Examples C1 through C6) showed far worse performance than Chitosan (Comparative Example C7).

### EXAMPLE 2

Permeation of SS using Example Polymer P7 on two different tissue samples. Permeation tests were performed as in Comparative Example 1. Results were as follows.

Permeation of SS

| Example: | C2-8 | C2-9 | 2-10 | 2-11 |
|---|---|---|---|---|
| Polymer: | none | Chitosan | P7 | P7 |
| Conc⁽¹⁾ | 0 | 0.1% | 0.5% | 0.5% |
| Remain⁽³⁾ | 102 | 63.5 | 31.9 | 53.6 |
| Perm⁽⁴⁾ | 3 | 30.3 | 22.3 | 28.5 |
| Peff⁽⁵⁾ | 0.35 | 3.5 | 2.6 | 3.3 |

⁽¹⁾% weight of polymer on total weight of solution
⁽³⁾weight % of SS remaining in donor solution based on total SS
⁽⁴⁾weight % or SS permeated through tissue based on total SS
⁽⁵⁾units are 10⁻⁶ cm/sec

Example polymer P7 performs far better than comparative polymers Compl, Comp2, and Comp3. Also, P7 performs comparably to Chitosan.

### EXAMPLE 3

### TEER Testing

TEER tests were performed on the sample reported in Example 2 above. Resistance drops from the initial value prior to permeation testing to the final value at the end of the permeation test; a larger drop indicates greater permeability. Results were as follows:

TEER tests

| Example: | C3-8 | C3-9 | 3-10 | 3-11 |
|---|---|---|---|---|
| Polymer: | none | Chitosan | P7 | P7 |
| Conc⁽¹⁾ | 0 | 0.1% | 0.5% | 0.5% |
| Resistance (ohms) | | | | |
| initial | 425 | 450 | 510 | 430 |
| final | 305 | 60 | 80 | 70 |

⁽¹⁾% weight of polymer on total weight of solution

P7 and Chitosan show far greater drop in resistance (and therefore a greater tendency to assist permeation) than does the buffer solution alone.

### EXAMPLE 4

### Tissue Viability Testing

After performing a permeation test as described above, tissues were given the viability test as described above. The samples contained SS. One sample had Triton™ X-100 surfactant at a level of 0.2% by weight. Results were as follows:

Viability Test

| Example: | C4-8 | 4-10 | 4-12 |
|---|---|---|---|
| Excipient: | PBS | P7 | Surfactant |
| Conc⁽¹⁾ | 0 | 0.5% | 0.2% |
| Viability (%) | 100 | 110 | 1 |

⁽¹⁾% weight of polymer on total weight of solution

The sample with surfactant had low viability. It is considered that the surfactant is likely to enhance permeation but cause a degradation of cell viability. The sample with P7 showed good permeability (as demonstrated above in a previous example) and good viability.

### EXAMPLE 5

### Membrane Recovery by the TEER Method

Samples were also tested for recovery in the TEER method. Samples had 2 mg/L SS. Results were as follows:

TEER Recovery test

| Example: | C5-12 | C5-13 | 5-14 |
|---|---|---|---|
| Excipient: | surfactant⁽⁶⁾ | none | P7 |
| Conc⁽¹⁾ | 0.2% | 0 | 0.2% |
| Resistance (ohms) | | | |
| initial | 800 | 710 | 510 |
| at 4 hours permeation | 1 | 580 | 80 |
| 24 hours after permeation | 2 | 530 | 610 |

⁽¹⁾% weight of polymer on total weight of solution
⁽⁶⁾Triton ™ X-100 surfactant described above

In Example 5, only example 5-14 with polymer P7 shows both (1) a drop in TEER at 4 hours permeation (which demonstrates good permeability) and good recovery of TEER 24 hours later (which demonstrates that the membranes recover from the treatment without permanent damage).

### EXAMPLE 6

### Permeation Testing of Formulations Using Example Polymer P7

Permeation tests were performed as in Comparative Example 1. Two different batches of example polymer P7 were used, labeled P7-1 and P7-2. Results were as follows.

Permeation of SS

| Example: | 6-15 | 6-16 | 6-17 | 6-18 | 6-19 | 6-20 | 6-21 | 6-22 | 6-23 |
|---|---|---|---|---|---|---|---|---|---|
| Polymer: | P7-2 | P7-2 | P7-1 | P7-1 | P7-1 | P7-1 | P7-2 | P7-2 | P7-1 |
| Tissue⁽⁷⁾ | S1 | S2 | S1 | S2 | S1 | S2 | S1 | S2 | — |
| Conc⁽¹⁾ | 0.5 | 0.5 | 0.2 | 0.2 | 0.2 | 0.2 | 0.2 | 0.2 | 0.02 |
| day | 1 | 1 | 2 | 2 | 3 | 3 | 3 | 3 | 3 |
| Remain⁽³⁾ | 31.9 | 53.6 | 60.0 | 88.7 | 15.5 | 16.7 | 15.5 | 11.9 | 44.2 |
| Perm⁽⁴⁾ | 22.3 | 28.5 | 37.5 | 14.1 | 73.6 | 55.6 | 72.5 | 72.5 | 44.2 |
| Peff⁽⁵⁾ | 2.6 | 3.3 | 4.3 | 1.6 | 8.5 | 6.4 | 8.4 | 8.4 | 5.1 |

⁽¹⁾% weight of polymer on total weight of solution
⁽³⁾weight % of SS remaining in donor solution based on total SS
⁽⁴⁾weight % or SS permeated through tissue based on total SS
⁽⁵⁾units are 10⁻⁶ cm/sec
⁽⁷⁾Pairs of examples (such as 6-15 and 6-16) that are identical except for “tissue” are replicate examples performed on two tissue samples that were different from each other. Each example was performed on a separate individual tissue sample; therefore, for example, the tissue “S1” of 6-15 is not the same tissue sample as “S1” of 6-17.

### EXAMPLE 7

Permeation testing of various example polymers. Further permeation testing was conducted as in Comparative Example 1. Two batches of P7 were used: P7-1 and P7-2. Results were as follows:

Permeation of SS

| Example: | 7-24 | 7-25 | 7-26 | 7-27 | 7-28 | 7-29 |
|---|---|---|---|---|---|---|
| Polymer: | P1 | P2 | P3 | P4 | P5 | P6 |
| Conc⁽¹⁾ | 0.2 | 0.2 | 0.2 | 0.2 | 0.2 | 0.2 |
| Remain⁽³⁾ | 20.2 | 36.5 | 16.5 | 38.3 | 22.5 | 33.8 |
| Perm⁽⁴⁾ | 56.6 | 44.2 | 39.5 | 40.4 | 34.9 | 23.1 |
| Peff⁽⁵⁾ | 6.6 | 5.1 | 4.6 | 4.7 | 4.0 | 2.7 |

Permeation of SS

| Example: | 7-30 | 7-31 | 7-32 | C7-33 | C7-34 |
|---|---|---|---|---|---|
| Polymer: | P7-1 | P8 | P7-2 | Chitosan | none |
| Conc⁽¹⁾ | 0.2 | 0.02 | 0.2 | 0.2 | 0 |
| Remain⁽³⁾ | 16.7 | 42.5 | 11.9 | 51.6 | 86.4 |
| Perm⁽⁴⁾ | 55.6 | 37.9 | 72.5 | 32.4 | 1.7 |
| Peff⁽⁵⁾ | 6.4 | 4.4 | 8.4 | 3.8 | 0.19 |

⁽¹⁾% weight of polymer on total weight of solution
⁽²⁾number of replicate samples tested. Results shown are averages over the replicates
⁽³⁾weight % of SS remaining in donor solution based on total SS
⁽⁴⁾weight % or SS permeated through tissue based on total SS
⁽⁵⁾units are 10⁻⁶ cm/sec

All of the example polymers P1 through P8 show significant improvement to permeability over the control sample that has no polymer.

1. A method of delivering a physiologically active agent into a tissue and/or a bloodstream of a living body, said method comprising applying an aqueous solution to a mucosal surface and allowing the physiologically active agent to permeate through the mucosal surface, wherein the aqueous solution comprises:
(a) a cationic polymer dissolved in water, wherein said cationic polymer comprises a hydrophobic quaternary ammonium group covalently attached to a hydroxyethyl cellulose polymer backbone and optionally further comprises a non-hydrophobic quaternary ammonium group covalently attached to the hydroxyethyl cellulose polymer backbone, and
(b) one or more physiologically active agents,
wherein, if the non-hydrophobic quaternary ammonium group is present, the molar ratio of the non-hydrophobic quaternary ammonium group to the hydrophobic quaternary ammonium group is from 0:1 to 0.1:1.

2. The method of claim 1 wherein the cationic polymer has no non-hydrophobic quaternary ammonium group covalently attached to the hydroxyethyl cellulose polymer backbone.

3. The method of claim 1 wherein said mucosal surface is in a vagina, a mouth, an eye, an ear, an esophagus, a stomach, intestines, or a nasal cavity.

4. The method of claim 1 wherein said mucosal surface is in a nasal cavity.

5. The method of claim 1 wherein the physiologically active agent is a drug.

6. The method of claim 1 wherein the amount of said cationic polymer is 0.01% to 10% by weight based on the weight of said aqueous solution.

7. The method of claim 1 wherein said cationic polymer comprises repeat units of Structure 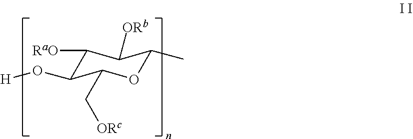
wherein n is 10 or higher; wherein R^{a}, R^{b}, and R^{c} is each H or [CH₂CH₂O]ₓ—R¹, and one or more of R^{a}, R^{b}, and R^{c} is [CH₂CH₂O]ₓ—R¹; wherein each x is chosen from 0, 1, 2, 3, or 4, and at least one repeat unit has at least one of R^{a}, R^{b}, and R^{c} in which x is 1, 2, 3, or 4; wherein each R¹ in R^{a}, R^{b}, and R^{c} is H or Structure III, and at least one R¹ is Structure III: 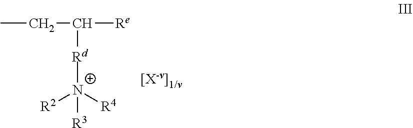
wherein —R^{d}— is a bivalent organic group, and —R^{e} is either a hydrogen atom or a hydroxyl (OH) group; wherein —R² and —R³ is each an alkyl group with 3 or fewer carbon atoms;
wherein —R⁴ is an alkyl group with 10 or more carbon atoms; and wherein X is an anion of valence v.

8. The method of claim 7 wherein —R^{d}— is —CH₂—; —R² and —R³ is each methyl; and —R⁴ is an alkyl group with 12 or more carbon atoms.

9. The method of claim 7 wherein —R^{a} and —R^{b} are —H, —R^{c} is —[CH₂CH₂O]ₓ—R′, R¹ is Structure III, and x is 1 or 2.

10. The method of claim 7 wherein X is a halide anion.

11. The method of claim 7 wherein said cationic polymer has cationic degree of substitution of 0.01 or higher.

12. The method of claim 7 wherein said cationic polymer has cationic degree of substitution of 0.02 or higher.

13. The method of claim 7 wherein said cationic polymer has cationic degree of substitution of 0.05 or higher.

14. The method of claim 1 wherein said cationic polymer has weight-average molecular weight (Mw) of 100,000 or higher.

15. The method of claim 1 wherein said cationic polymer has weight-average molecular weight (Mw) of from 100,000 to 500,000.

16. The method of claim 1 wherein the aqueous solution is a buffer solution and further comprises an inorganic salt.

17. The method of claim 16 wherein the aqueous solution is a phosphate buffered saline solution.

18. The method of claim 1 wherein the aqueous solution is a liquid aqueous solution.

19. The method of claim 18 wherein the viscosity of the liquid aqueous solution is 300 mPa·s or less when measured by steady shear viscometry using cone and plate at 10 sec⁻¹ at 25° C.

20. The method of claim 18 wherein the viscosity of the liquid aqueous solution is 30 mPa·s or less when measured by steady shear viscometry using cone and plate at 10 sec⁻¹ at 25° C.
